(19) United States
(12) Patent Application Publication
(10) Pub. No.: US 2004/0152791 A1
(43) Pub. Date: Aug. 5, 2004

(54) Catalyst

(76) Inventors: Andre Johan Van Zyl, Parys (ZA); Jacobus Lucas Visagie, Sasolburg (ZA); Herman Preston, Vaalpark (ZA); Masikana Millan Mdleleni, Sasolburg (ZA); Humphrey Thulani Dlamini, Vanderbijlpark (ZA); Thato Rose Motjope, Vanderbijlpark (ZA)

Correspondence Address: Browning Bushman P.C., Suite 1800, 5718 Westheimer, Houston, TX, 77057-5771, US

(21) Appl. No.: 10/720,302

(22) Filed: Nov. 24, 2003

(30) Foreign Application Priority Data
Nov. 25, 2002 (ZA) 2002/9563

(51) Int. Cl. C07C 27/06

(52) U.S. Cl. 518/720; 502/338

(57) ABSTRACT

This invention relates to a new method for producing an iron-based Fischer-Tropsch catalyst composition wherein the main iron phase is ferrihydrite in a precipitation reaction. It has been found that the reduction of Fe(III) species to Fe(II), and thus an increase in the Fe(II)/Fe(III) ratio, prior to precipitation affects the crystallite size of the crystallite particles of the catalyst, and enhances the olefin selectivity of the catalyst. The reduction of the Fe(III) species to Fe(II) can be attained in situ by the addition of a reduction agent (such as oxalic acid or formic acid) during the catalyst preparation method.

### BACKGROUND OF THE INVENTION

This invention relates to iron-based catalysts, in particular to iron-based catalysts and their use in the conversion of synthesis gas (CO and H₂) to alcohols and olefins.

International patent publication no. WO 01/89689 discloses an iron-based Fischer-Tropsch catalyst composition wherein the iron phase is ferrihydrite. The catalyst composition includes natural promoters which may be selected from manganese or chromium or a mixture thereof and chemical promoters selected from magnesium zinc, copper and alkaline or alkali earth metals. The catalyst is best bound to refractory oxide support. The catalyst composition is formed by preparing a solution containing Fe ions and nitrates of Zn, Mn and Cu. This solution is reacted with a solution containing a base, such as KOH, to form a precipitate wherein the main iron phase of the precipitate is ferrihydrite. The precipitate is then washed, dried and bound with a refractory metal oxide. According to the specification, the catalyst composition produces significant yield of higher paraffins, olefins and alcohols.

It is an object of this invention to provide an improved method of producing an iron-based Fischer-Tropsch catalyst composition which has greater selectivity towards olefins.

### SUMMARY OF THE INVENTION

This invention relates to a method for producing a iron-based Fischer-Tropsch catalyst composition, the catalyst composition having a high olefin selectivity when used in a Fischer-Tropsch reaction.

According to the invention there is provided a method for producing an iron-based Fischer-Tropsch catalyst composition (typically a composition where the main iron phase is ferrihydrite) in a precipitation reaction of Fe ions in solution, wherein Fe(III) ions in solution are reduced to Fe(II) and thus the ratio of Fe(II)/Fe(III) in the solution is increased prior to precipitation of the catalyst. The ratio of Fe(II)/Fe(III) may be increased by introducing a stoichiometric amount of a reduction agent such as oxalic acid or formic acid to the solution during the catalyst preparation method.

By “iron-based” is meant that Fe makes up at least 30% (by mass) of the composition. The term “the main iron phase is ferrihydrite” means that at least 75% of the iron phase is ferrihydrite, as determined by X-ray diffraction using Co K alpha radiation. The preferred catalyst compositions exhibit hyperfine interaction parameters similar to those of ferrihydrite, as determined by Mössbauer absorption spectroscopy (MAS).

A “structural promoter” is a chemical species/element that helps to stabilise the ferrihydrite phase of the catalyst.

A typical method for preparing an iron-based catalyst pre-cursor wherein the main iron phase is ferrihydrite, includes the following steps:

1. preparing a first solution in a polar solvent, the solution containing Fe(III) ions and a reduction agent such as oxalic acid or formic acid to reduce Fe(III) ions to Fe(II) ions;

2. adding a precipitation agent, typically a base, to the solution to form a catalyst precipitate wherein the main iron phase is ferrihydrite;

3. washing the precipitate;

4. drying, typically spray-drying, the washed precipitate; and

5. calcining the dried precipitate.

The first solution may be formed by dissolving a ferric salt, such as Fe nitrate, in the polar solvent.

Typically, the ions of structural promoters such as Mn, Cu, Zn, Cd, Ni, Co, Al and chemical promoters such as Zn, Mg, Cu, Cr, Ru, Pd, Rh or and alkaline or alkali earth metals such as K, Na and La are included in the first solution.

Typically, the first solution includes iron nitrate, manganese nitrate, copper nitrate, zinc nitrate and aluminium nitrate.

The preferred base is KOH, however NaOH and Na₂CO₃ and K₂CO₃ can also be used.

Preferably, the step 2 is carried out at a pH of 7-9, most preferably a pH of 8, and at a temperature of 50° C.-80° C., most preferably 68° C.-72° C.

According to a further aspect of the invention there is provided a method for producing higher paraffins, alcohols and particularly olefins selectively, by reacting hydrogen with carbon monoxide in the presence of a catalyst substantially as described herein above.

### BRIEF DESCRIPTION OF THE DRAWINGS

FIG. 1 is a graph showing the crystallite size vs mole ratio of oxalic acid/Fe(III) catalysts prepared by using zero and different amounts of oxalic acid;

FIG. 2 is a graph showing the activities for catalysts prepared by using different amounts of oxalic acid;

FIG. 3 shows the alcohol selectivity for catalysts prepared using different amounts of oxalic acids; and

FIG. 4 shows the olefin selectivity of catalysts prepared using different amounts of oxalic acids

### DESCRIPTION OF EMBODIMENTS

This invention relates to a new method for producing an iron-based Fischer-Tropsch catalyst composition wherein the main iron phase is ferrihydrite in a precipitation reaction. It has been found that the reduction of Fe(III) species to Fe(II), and thus an increase in the Fe(II)/Fe(III) ratio, prior to precipitation affects the crystallite size of the crystallite particles of the catalyst, and enhances the olefin selectivity of the catalyst. The reduction of the Fe(III) species to Fe(II) can be attained in situ by the addition of a reduction agent (such as oxalic acid or formic acid) during the catalyst preparation method.

The catalyst composition of the invention may be produced in a continuous precipitation method by making a first acidic solution containing Fe nitrate (Fe (NO₃)₃.9H₂O), Mn nitrate (Mn (NO₃)₂.4H₂O), Zn nitrate Zn (NO₃)₂.6H₂O), Cu nitrate Cu (NO₃)₂.3H₂O), Al nitrate (Al (NO₃)₃.9H₂O) and a reduction agent in the form of oxalic acid, and heating the solution to 65° C. A second basic solution, containing 25%, by mass, KOH and at a temperature of 45° C. is then added to the first solution in a precipitation vessel. The rate at which the second solution is added to the first solution is adjusted so that the pH is maintained at a range of 7-9, preferably approximately 8 and the temperature at 50° C.-80° C., preferably approximately 68° C.-72° C. The addition of the second solution to the first solution causes the formation of a precipitate, which is the catalyst composition of the invention. The precipitate is then filtered, and washed and the filter cake then reslurried, optionally bound with SiO₂ or Al₂O₃, and spray-dried at an inlet temperature of 260° C. and an outlet temperature of 120° C. Thereafter, on-spec catalyst is calcined at 450° C. for 16 hours.

Table 1 below shows the elemental composition of a catalyst OLE 135A which was prepared using no oxalic acid, catalysts OLE 135B-G which were prepared using oxalic acid and catalyst OLE 135L which was prepared using formic acid. The percent oxalic acid is calculated in terms of Fe(III), i.e. 10% oxalic acid=10 moles/100 moles Fe(III).

TABLE 1

| | | Mole ratio | | | | | |
|---|---|---|---|---|---|---|---|
| | % oxalic | of oxalic | gZn/100 g | gMn/100 g | gCu/100 g | gK₂O/100 g | gSiO₂/100 g |
| Catalyst | acid | acid:Fe(III) | Fe | Fe | Fe | Fe | Fe |
| OLE | 0 | 0 | 10.62 | 25.52 | 6.7 | 0.45 | 14.78 |
| 135A | | | | | | | |
| OLE | 20 | 0.2 | 10.84 | 24.35 | 6.97 | 1.37 | 8.09 |
| 135B | | | | | | | |
| OLE | 40 | 0.4 | 11.81 | 26.58 | 7.22 | 1.31 | 8.15 |
| 135C | | | | | | | |
| OLE | 60 | 0.6 | 10.98 | 22.76 | 6.22 | 1.09 | 9.74 |
| 135D | | | | | | | |
| OLE | 80 | 0.8 | 10.87 | 22.3 | 6.11 | 1.37 | 12.2 |
| 135E | | | | | | | |
| OLE | 100 | 1 | 10.18 | 18.16 | 5.16 | 2.02 | 11.74 |
| 135F | | | | | | | |
| OLE | 10 | 0.1 | 12.62 | 27.12 | 8.13 | 0.5 | 11.37 |
| 135G | | | | | | | |
| OLE | 10 | 0.1 | 12.31 | 28.74 | 7.98 | 2.23 | 13.76 |
| 135L | | formic acid | | | | | |

Table 2 below shows the surface area and pore volumes of the catalysts identified in Table 1 above.

TABLE 2

| Catalyst | Surface Area m²/g | Pore Volume cm³/g |
|---|---|---|
| OLE A | 95.78 | 0.40 |
| OLE B | 97.79 | 0.45 |
| OLE C | 75.34 | 0.42 |
| OLE D | 144.68 | 0.42 |
| OLE E | 125.38 | 0.45 |
| OLE F | 144.13 | 0.42 |
| OLE G | 102.6 | 0.47 |
| OLE L | 111.1 | 0.52 |

The catalyst composition according to the invention formed with a sufficient amount of a reduction agent is iron-based, the main iron phase is ferrihydrite, and the catalyst has a surface area of 100-300 m²/g and a pore volume of 0.4 to 0.52.

The reduction agent partly reduces Fe(II) to Fe(II) causing a higher Fe(II)/Fe(II) ratio. This reaction is illustrated as follows:

Fe(III)(NO₃)₃(s)+H₂C₂O₄(s)→Fe(II)C₂O₄(s)+HNO₃(aq)+H₂+CO₂

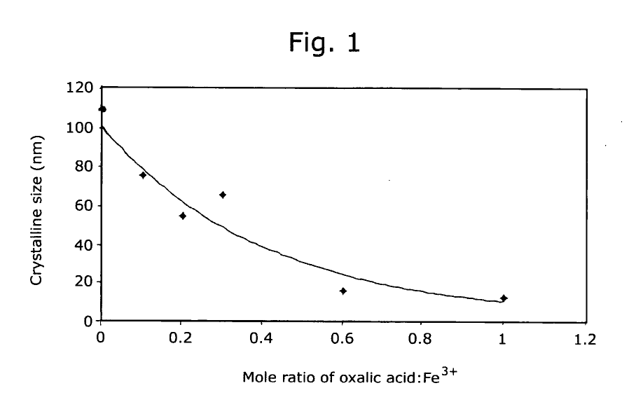

FIG. 1 attached shows the mean crystallite size vs mole ratio of oxalic acid/Fe(III). As will be seen from FIG. 1, it has been found that crystallite size of the catalyst increases with a decrease in the amount of oxalic acid added to the first solution. Table 2 shows that catalysts prepared with the introduction of 60% or more oxalic acid, (i.e. where the mole ratio of oxalic acid to Fe(III) is 0.6 or greater) have higher surface areas than the catalysts prepared with lower amounts of oxalic acid.

Catalysts prepared according to the invention were then tested. 20 g of catalyst is loaded in a Fischer-Trospch reactor in 350 g molten wax, followed by in-situ reduction at 240° C., 20 bar and hydrogen gas hourly space velocity of 6000 ml(n)/g cat/hr for 16 hours. After reduction the temperature was lowered to 200° C. and Arge Pure Gas (APG, approx. 75% syngas) introduced at 4500 syngas space velocity for one hour (This stage is known as catalyst conditioning. APG is introduced at a lower P & T and then gradually increased to synthesis conditions to avoid catalyst breakage). The temperature and pressure were then increased to 240° C. and 45 bar respectively. After the first period analyses space velocities were adjusted to operate at 40% conversion. Gas samples were analysed by TCD (Thermal Conductivity detector is used to analysis for CO₂, CO, H₂, CH₄, Ar, N₂ (permanent gases)) and FID GC (Flame lonisation detector is used to analyse all gaseous products (alcohols, paraffins, olefins, etc)) and the oils and water by FID GC (Flame lonisation detector used to analyse liquid products (alcohols, paraffins, olefins, water, etc)) to calculate the product distribution.

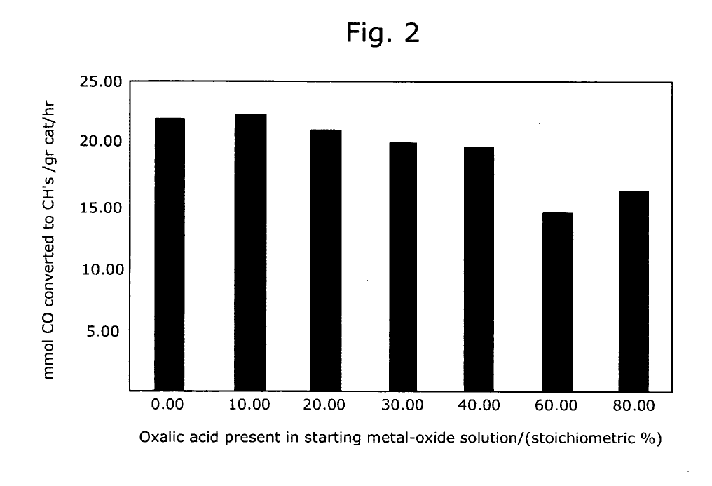

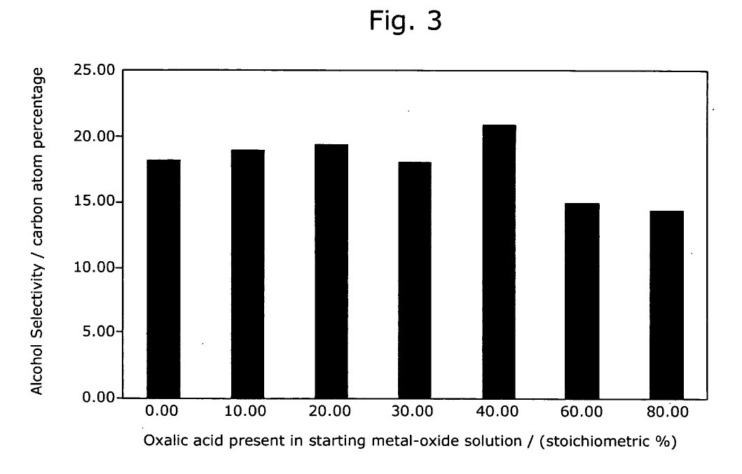

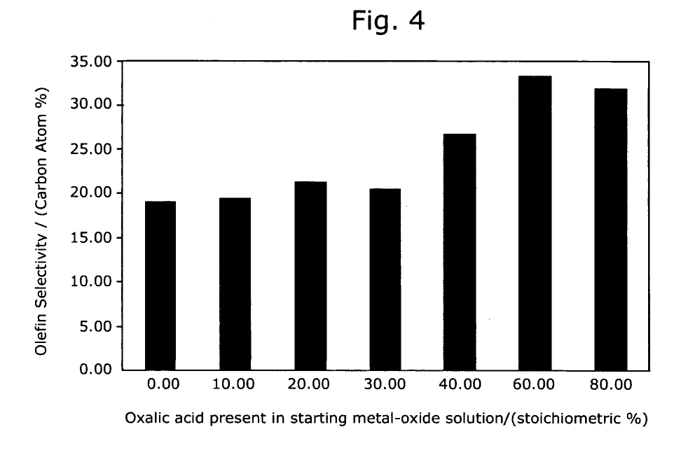

Results of tests are shown in the attached FIGS. 2, 3 and 4. As can be seen from FIG. 2, the amount of 10% to 40% reduction agent did not have a large negative effect on the activity of the catalyst. It will be seen from FIG. 3 that the inclusion of from 10% to 40% reduction agent did not have a negative effect on the alcohol selectivity of the catalyst, with a slight increase in selectivity with the addition of 40%. FIG. 4 however shows that the inclusion of more than 40% or more reduction agent leads to a large increase in the selectivity of the catalyst for olefins.

The invention will now be explained in more detail with reference to the following non-limiting Examples:

### Example 1

### Preparation of OLE 135A-G

Catalyst samples OLE 135A-G were prepared via the continuous precipitation method, where nitrates of Fe, Zn, Mn, Cu and oxalic acid were reacted with 25% KOH solution. The first step of preparation entailed the addition of various amounts of oxalic acid to the metal salt solutions heated to ˜80° C. The KOH solution was kept at ˜45° C. These two solutions were then co-fed into the precipitation vessel. The rate at which these solutions were fed was adjusted such that the precipitation pH was ˜8 and temperature was between 65-70° C. After precipitation, the precipitates were filtered and washed to ˜2 mS. The precipitates were then reslurried, bound with SiO₂ (10 g/100 g Fe). The final catalysts were spray dried at constant pump rate using the following conditions:

| | |
|---|---|
| Inlet Temperature = | 260° C. |
| Outlet temperature = | 120° C. |
| Air flow = | 65% of maximum |

Catalysts were calcined at 450° C. for 16 h.

The starting materials used for the preparation of these catalysts is presented in Table 3 and the final composition (mass %) is reported in Table 4.

TABLE 3
Raw materials of the preparation of OLE 135A-G

| | Raw materials used [g] | | | | | | |
|---|---|---|---|---|---|---|---|
| | | | | | | Silica | |
| | | | | | | sol | 25% |
| | Fe | Zn | Mn | Cu | Oxalic | 25% | KOH |
| Catalyst | nitrate | nitrate | nitrate | nitrate | acid | SiO₂ | solution |
| OLE | 1787 | 143 | 399 | 68 | 0 | 90 | 1050 |
| 135A | | | | | | | |
| OLE | 1787 | 143 | 399 | 68 | 6.71 | 90 | 1050 |
| 135B | | | | | | | |
| OLE | 1787 | 143 | 399 | 68 | 13.4 | 90 | 1050 |
| 135C | | | | | | | |
| OLE | 1787 | 143 | 399 | 68 | 20.1 | 90 | 1050 |
| 135D | | | | | | | |
| OLE | 1787 | 143 | 399 | 68 | 26.8 | 90 | 1050 |
| 135E | | | | | | | |
| OLE | 1787 | 143 | 399 | 68 | 33.1 | 90 | 1050 |
| 135F | | | | | | | |
| OLE | 1787 | 143 | 399 | 68 | 3.36 | 90 | 1050 |
| 135G | | | | | | | |

TABLE 4
Final elemental composition of OLE 135A-G (mass %)

| Catalyst | % Fe | % Cu | % Mn | % Zn | % SiO2 | % K₂O |
|---|---|---|---|---|---|---|
| OLE | 46 | 3.08 | 11.5 | 5.06 | 11.6 | 0.21 |
| 135A | | | | | | |
| OLE | 45 | 3.15 | 10.8 | 4.95 | 6.12 | 0.62 |
| 135B | | | | | | |
| OLE | 45 | 3.15 | 11.7 | 5.31 | 6.12 | 0.59 |
| 135C | | | | | | |
| OLE | 43 | 2.67 | 9.46 | 4.73 | 7.09 | 0.47 |
| 135D | | | | | | |
| OLE | 43 | 2.63 | 9.46 | 4.73 | 8.77 | 0.59 |
| 135E | | | | | | |
| OLE | 44 | 2.27 | 7.92 | 4.4 | 8.75 | 0.88 |
| 135F | | | | | | |
| OLE | 44 | 3.52 | 11.9 | 5.54 | 8.45 | 0.22 |
| 135G | | | | | | |

### EXAMPLE 2

### Use of an Alternate Reducing Agent

For OLE 135L, formic acid with a mole ratio of 0.1 was used instead of oxalic acid. The preparation procedure was exactly as described in Example 1.

The starting materials used for this preparation is the same as that presented in Table 1 (for Fe, Cu, Mn, Zn and SiO2). The amount of formic acid used to obtain a mole ratio of 0.1 was 2.66 g. This translated to ca. 0.06% (mole %).

The final composition of OLE 135L in mass % is presented in Table 5.

TABLE 5
Final elemental composition of OLE 135L(mass %)

| Catalyst | % Fe | % Cu | % Mn | % Zn | % SiO2 | % K₂O |
|---|---|---|---|---|---|---|
| OLE 135L | 36 | 2.88 | 10.4 | 4.43 | 8.42 | 36 |

We claim:

1. A method for producing an iron-based Fischer-Tropsch catalyst composition in a precipitation reaction, wherein Fe(III) ions in solution are reduced to Fe(II) thereby increasing the Fe(II)/Fe(II) ion ratio, prior to the precipitation of the catalyst.

2. The method according to claim 1, wherein the Fe(III) ions are reduced to Fe(II) by the introduction of a reduction agent to the solution.

3. The method according to claim 1 or 2, wherein the reduction agent is oxalic acid or formic acid.

4. The method according to claim 3, wherein the reduction agent is oxalic acid.

5. The method according to claim 4, wherein the mole ratio of oxalic acid to Fe(III) is 0.4 or greater.

6. A method for preparing an iron-based catalyst pre-cursor including the following steps:
1. preparing a first solution in a polar solvent, the solution containing Fe(III) ions and reducing Fe(III) ions to Fe(II) ions;
2. adding a precipitation agent to the solution to form a catalyst precipitate;
3. washing the precipitate;
4. drying, typically spray-drying, the washed precipitate; and
5. calcining the dried precipitate.

7. The method according to claim 6, wherein the first solution is formed by dissolving a ferric salt in the polar solvent.

8. The method according to claim 7, wherein the ferric salt is Fe nitrate.

9. The method according to any one of claims 6-8, wherein the Fe(III) ions are reduced to Fe(II) ions by introducing a reduction agent into step 1.

10. The method according to claim 9, wherein 40% or more reduction agent, calculated in terms of Fe(III), is included in the first solution in step 1.

11. The method according to claim 10, wherein the reduction agent is oxalic acid or formic acid.

12. The method according to claim 11, wherein the reduction agent is oxalic acid.

13. The method according to claim 6, wherein the ions of structural promoters including Mn, Cu, Zn, Cd, Ni, Co, Al and chemical promoters including Zn, Mg, Cu, Cr, Ru, Pd, Rh or and alkaline or alkali earth metals including K, Na and La are included in the first solution.

14. The method according to claim 13, wherein the first solution in step 1 includes iron nitrate, manganese nitrate, copper nitrate, zinc nitrate and aluminium nitrate.

15. The method according to claim 6, wherein the precipitation agent in step 2 is a base.

16. The method according to claim 15, wherein the base is KOH.

17. The method according to claim 6, wherein step 2 is carried out at a pH of 7-9.

18. The method according to claim 17, wherein step 2 is carried out at a pH of 8.

19. The method according to claim 6, wherein step 2 is carried out at a temperature of 50° C.-80° C.

20. The method according to claim 19, wherein step 2 is carried out at a temperature of 68° C.-72° C.

21. The method according to claim 6, wherein the main iron phase of the catalyst composition is ferrihydrite.

22. A method for producing higher olefins selectively, by reacting hydrogen with carbon monoxide in the presence of a catalyst produced in a method according to claim 1.
